(19) United States
(12) Patent Application Publication
(10) Pub. No.: US 2012/0088823 A1
(43) Pub. Date: Apr. 12, 2012

(54) COMPOSITION AND METHOD FOR SCALP AND HAIR TREATMENT

(75) Inventors: Masato IINO, Yokohama-shi (JP); Satoshi YAMAKI, Yokohama-shi (JP); Satoshi NAKANISHI, Tokyo-to (JP); Masahiro TAJIMA, Tokyo-to (JP); Ryuji UENO, Potomac, MD (US)

(73) Assignee: R-TECH UENO, LTD., Tokyo (JP)

(21) Appl. No.: 13/329,639

(22) Filed: Dec. 19, 2011

Related U.S. Application Data
(62) Division of application No. 12/534,775, filed on Aug. 3, 2009, now abandoned.
Application No. 12/534,775 is a continuation of application No. 11/794,120, filed on Jun. 25, 2007, now abandoned.
(60) Provisional application No. 60/639,845, filed on Dec. 29, 2004.

(30) Foreign Application Priority Data
Feb. 10, 2005 (JP) 2005-034763

(51) Int. Cl. A61K 31/357; A61P 17/14; A61K 31/215

(52) U.S. Cl. 514/452; 514/530; 514/467

(57) ABSTRACT

Provided is a composition for scalp and hair treatment, comprising a menthol derivative and a prostaglandin compound having two hetero atoms at the 15 position. The composition of the present invention is effective not only for preventing dandruff and itchy scalp but also for preventing or treating hair loss, baldness, or hair thinness.

### TECHNICAL FIELD

This invention is directed to the field of hair and scalp treatment. In more detail, the invention is directed to a hair and scalp treating composition which can be provided as medical, medicated cosmetic or cosmetic product. The invention also relates to a method for treating the scalp and hair.

### ART RELATED

Various troubles concerning scalp and/or hair are increasing in this stressful aging society. Demands for scalp and/or hair care products for treating those troubles are rapidly increasing.

Many products for treating scalp and/or hair, including hair cosmetics and hair growth promoting formulae suitable for various hair and scalp conditions have been developed. For example, products for preventing hair loss by preventing dandruff and itching have been developed.

In general, known causes of baldness, alopecia, hair loss, hair thinness, dandruff and itchy scalp include activation of male hormone at certain organs such as hair gland, overproduction of sebum, generation of peroxide lipids, decrease of blood circulation in the follicle and stresses. Insufficient nutrition to follicle fails to grow strong and beautiful hair and may cause thinner hairs. In addition, decrease of blood circulation in follicle may cause insufficient nutrition as well as impairment of waste excreting function. In view of the above described mechanisms concerning hair loss, improving the turn over of scalp stratum corneum, suppressing hyper sebum production as well as promoting scalp blood circulation have been believed as keys for developing agents for treating troubles concerning scalp and/or hair.

Conventional products for scalp and hair treatment, in general, have been manufactured by combining agents which is effective for removing or alleviating one or more causes of baldness or hair loss. For example, vitamins such as vitamin B and vitamin E, amino acids such as serine and methionine, nicotinic acids, Swertia japonica extract, vasodilating agent such as acetylcholine derivatives, anti-inflammatory agent such as erythrorhizon, female hormone such as estradiol, and skin functioning agent such as cepharathin are used for manufacturing composition for treating and preventing baldness, hair loss and/or hair thinness. (Japanese Patent Laid Open Nos. H2-48516, H5-255044, H7-206647, H7-277930 and 2001-288047)

One of the inventors of the invention had found that prostaglandin compound having two hetero atoms at the 15-position has a hair growth promoting activity and filed an International application (WO2005/013928, the disclosure of the application is herein incorporated by reference).

### SUMMARY OF THE INVENTION

An object of the invention is to provide a composition for hair and scalp treatment, which is effective not only for preventing dandruff and itchy scalp but also for promoting hair growth and/or preventing hair loss, and is excellent in stability and safety. Further object of the present invention to provide a method for treating the scalp and/or hair of a subject having suffered from scalp and/or hair troubles.

The present invention provides a composition for scalp and/or hair treatment, which comprises a menthol derivative and a prostaglandin compound having two hetero atoms at the 15 position.

The present invention also provides a method for treating the scalp and/or hair of a subject in need thereof, which comprises applying an effective amount of a menthol derivative and a prostaglandin compound having two hetero atoms at the 15 position topically to the scalp and/or hair of the subject.

### DETAILED DESCRIPTION OF THE INVENTION

The nomenclature of the PG compounds used in the specification and claims is based on the numbering system of the prostanoic acid represented in the following formula (A).

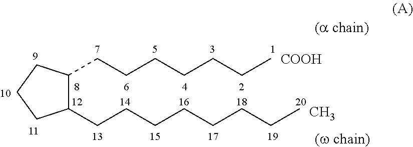

The formula (A) shows a basic skeleton of the C-20 carbon atoms, but the present invention is not limited to those having the same number of carbon atoms. In the formula (A), the numbering of the carbon atoms which constitute the basic skeleton of the PG compounds starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of the carboxy group (C-1). Similarly, when the number of carbon atoms is decreased in the ω-chain, the number is deleted in the order starting from position 20; and when the number of carbon atoms is increased in the ω-chain, the carbon atoms beyond position 20 are named as substituents. Stereochemistry of the compounds is the same as that of the above formula (A) unless otherwise specified.

In general, each of the terms PGD, PGE and PGF represents a PG compound having hydroxy groups at positions 9 and/or 11, but in the present specification, these terms also include those having substituents other than the hydroxy group at positions 9 and/or 11. Such compounds are referred to as 9-dehydroxy-9-substituted-PG compounds or 11-dehydroxy-11-substituted-PG compounds. A PG compound having hydrogen in place of the hydroxy group is simply named as 9- or 11-dehydroxy-PG compound.

As stated above, the nomenclature of the PG compounds is based on the prostanoic acid skeleton. However, in case the compound has a similar partial structure as a prostaglandin, the abbreviation of “PG” may be used. Thus, a PG compound of which α-chain is extended by two carbon atoms, that is, having 9 carbon atoms in the α-chain is named as 2-decarboxy-2-(2-carboxyethyl)-PG compound. Similarly, a PG compound having 11 carbon atoms in the α-chain is named as 2-decarboxy-2-(4-carboxybutyl)-PG compound. Further, a PG compound of which ω-chain is extended by two carbon atoms, that is, having 10 carbon atoms in the ω-chain is named as 20-ethyl-PG compound. These compounds, however, may also be named according to the IUPAC nomenclatures.

Examples of the analogs (including substituted derivatives) or derivatives include a PG compound of which carboxyl group at the end of α-chain is esterified; a compound of which α-chain is extended; physiologically acceptable salt thereof; a compound having a double bond at 2-3 position or a triple bond at position 5-6, a compound having substituent(s) at position 3, 5, 6, 16, 17, 18, 19 and/or 20; and a compound having lower alkyl or a hydroxy (lower) alkyl group at position 9 and/or 11 in place of the hydroxy group.

According to the present invention, preferred substituents at position 3, 17, 18 and/or 19 include alkyl having 1-4 carbon atoms, especially methyl and ethyl. Preferred substituents at position 16 include lower alkyl such as methyl and ethyl, hydroxy, halogen atoms such as chlorine and fluorine, and aryloxy such as trifluoromethylphenoxy. Preferred substituents at position 17 include lower alkyl such as methyl and ethyl, hydroxy, halogen atoms such as chlorine and fluorine, aryloxy such as trifluoromethylphenoxy. Preferred substituents at position 20 include saturated or unsaturated lower alkyl such as C1-4 alkyl, lower alkoxy such as C1-4 alkoxy, and lower alkoxy alkyl such as C1-4 alkoxy-C1-4 alkyl. Preferred substuents at position 5 include halogen atoms such as chlorine and fluorine. Preferred substituents at position 6 include an oxo group forming a carbonyl group. Stereochemistry of PGs having hydroxy, lower alkyl or hydroxy(lower)alkyl substituent at position 9 and/or 11 may be α, β or a mixture thereof.

Further, the above analogs or derivatives may be compounds having an alkoxy, cycloalkyl, cycloalkyloxy, phenoxy or phenyl group at the end of the co-chain where the chain is shorter than the primary PGs.

A preferred prostaglandin compound used in the present invention is represented by formula (I):

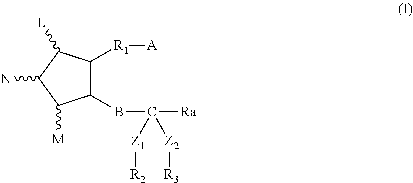

wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;

A is —CH₃, or —CH₂OH, —COCH₂OH, —COOH or a functional derivative thereof;

B is —CH₂—CH₂—, —CH═CH—or —C≡C—;

Z₁ and Z₂ are oxygen, nitrogen or sulfur,

R₂ and R₃ are optionally substituted lower alkyl, which is optionally linked together to form lower alkylene,

R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group.

A more preferred prostaglandin compound used in the present invention is represented by the formula (II):

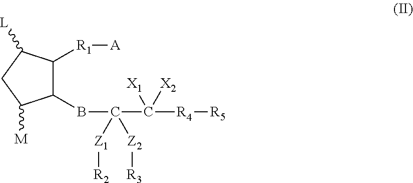

wherein L and M are hydrogen, hydroxy, halogen, lower alkyl, hydroxy(lower)alkyl, lower alkanoyloxy or oxo, wherein at least one of L and M is a group other than hydrogen, and the five-membered ring may have one or more double bonds;

A is —CH₃, or —CH₂OH, —COCH₂OH, —COOH or a functional derivative thereof;

B is —CH₂—CH₂—, —CH═CH— or —C≡C—;

Z₁ and Z₂ are oxygen, nitrogen or sulfur,

R₂ and R₃ are optionally substituted lower alkyl, which is optionally linked together to form lower alkylene,

X₁ and X₂ are hydrogen, lower alkyl, or halogen;

R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur;

R₄ is a single bond or lower alkylene; and

R₅ is lower alkyl, lower alkoxy, lower alkanoyloxy, cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-l-hydroxyethyl.

The term “lower alkanoyloxy” refers to a group represented by the formula RCO—O—, wherein RCO— is an acyl group formed by oxidation of a lower alkyl group as defined above such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydroxy and oxo, and especially, M and L are hydroxy groups which has a 5-membered ring structure of, so called, PGF type.

Preferred example of A is —COOH, its pharmaceutically acceptable salt, ester or amide thereof.

Preferred B is —CH₂—CH₂—, so called 13,14-dihydro type.

Preferred example of X₁ and X₂ is fluorine, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur.

Examples of R₁ include, for example, the following groups:

—CH₂—CH₂—CH₂—CH₂—CH₂—CH₂,

—CH₂—CH═CH—CH₂—CH₂—CH₂.

—CH₂—CH₂—CH₂—CH₂—CH═CH—,

—CH₂—C≡C—CH₂—CH₂—CH₂,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom.

Preferred Z₁ and Z₂ are oxygen.

R₂ and R₃ are preferably linked together to form C2 or C3 alkylene.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, any of isomers such as the individual tautomeric isomers, the mixture thereof, or optical isomers, the mixture thereof, a racemic mixture, and other steric isomers may be used in the same purpose.

In the present invention, preferred prostaglandin compounds are 13,14-dihydro-15,15-ethylenedioxy-20-ethyl-PGF_{2α} isopropyl ester, 13,14-dihydro-15,15-ethylenedioxy-17-phenyl-18,19,20-trinor-PGF_{2α} isopropyl ester, 13,14-dihydro-15,15-trimethylenedioxy-20-ethyl-PGF_{2α} isopropyl ester, 13, 14-dihydro-15,15-dimethoxy-20-ethyl-PGF_{2α} isopropyl ester and 13,14-dihydro-15,15-ethylenedioxy-20-ethyl-PGF_{2α} ethyl ester. Especially, 13,14-dihydro-15,15-ethylenedioxy-20-ethyl-PGF_{2α} ethyl ester is preferably used.

The amount of the prostaglandin compound in the composition of the present invention may be those effective for providing enough blood circulation promotion and/or scalp softening effects and those will not bring difficulties in manufacturing the product. The amount may be 0.0001-10.0 wt % (dry weight), preferably, 0.001-5.0 wt % per total weight of the composition.

The menthol derivative used in the present invention may be any menthols which are used in cosmetic or medical products such as 1-menthol and dl-menthol. Menthol derivatives are commercially available or may be obtained by extracting Mentha herbs such as pepper mint. Menthol derivatives may be used either alone or in combination.

The amount of the menthol derivative in the composition of the present invention may be those effective for providing anti-inflammatory, anti-skin roughness, anti-dandruff, anti-itching, hair growth promoting and/or hair loss preventing effects. In addition, the upper limit of the amount should be determined such that the composition will not provide unfavorable feeling of stimulation and will not bring difficulties in manufacturing the product.

The amount of menthol derivative in the composition of the present invention may be 0.001-5.0 wt %, preferably, 0.01-3.0 wt % per total amount of the composition.

The composition of the present invention may further comprise active ingredients, which are usually added to conventional compositions for scalp and/or hair treatment, unless the object of the present invention is impaired.

Examples of the active ingredients which may be added to the composition of the present invention include agents which can improve the blood circulation such as Swertia japonica extract, vitamin E or its derivatives, acetylcholine derivatives, cepharanthin, carpronium chloride, nicotinic acids and nicotinic acid derivatives and minoxidil; local stimulators such as capsicum tincture, nonylic acid vanilamide, nonylic acid vanillylamide, camphor, ginger tincture and nicotinic acid benzyl ester; anti-seborrhea agents such as pyridoxine or its derivatives, sulfur and vitamin B6; metabolic enhancer such as photosensitive pigment 301, placental protein, biotin, nicotinamide, vitamin B6 and its derivatives, biotin, pantothenic acid and its derivatives, cepharanthine, mono-nitroguaiacol, sodium mono-nitroguaiacol, 6-benzyl aminopurine, diisopropylamine dichloroacetate, hinokitiol, pentadecanoic acid monoglyceride, DADA, CTP and SD-35; antiphlogistics such as glycyrrhizic acid, glycyrrhetinic acid or their derivatives, adenosine or its derivatives, lithospermi radix extract, salicylic acid and its derivatives, octopirox, zinc oxide, allantoin, benzalkonium chloride, isopropyl methylphenol, camphors, ichthammol, guaiazulene, epsilon, aminocaproic acid, lysozyme chloride and diphenhydramine hydrochloride; 5α-reductase inhibitor such as oxendolone and finasteride; moisturizing agent such as glycerin, 1,3-butylene glycol, propylene glycol, dipropylene glycol, polyethylene glycol 400, polyethylene glycol 1500, polyethylene glycol 4000, polyethylene glycol 6000, glucam E-10, hyaluronic acid, bio-hyaluronic acid, sorbit solution, erythritol, maltitol, soluble collagen, chondroitin sulfate, polysaccharide from polianthes tuberosa, urea, trisaccharide, vitamin C phosphate ester calcium salt and pyrrolidone sodium carboxylate; female hormones such as estradiol and estrone; amino acids such as serine, methionine and tryptophan, vitamins such as vitamin A, B2, B12 and D, pantothenic acid or its derivative, and so on.

The plant extracts which are usually added to conventional compositions for scalp and/or hair treatment may also be added to the composition of the present invention. Examples of the plant extracts include those obtained from the following plants: Althaea officinalis, Coix seed, Rumex crispus, capsicum, aloe, wolfberry, mugwort, rice, Vitex rotundifolia, Rosmarinus, Drynaria Rhizome (Drynaria fortunei), broom, gentian, red rooted sage (Salvia miltiorrhiza), sponge cucumber, balloon flower, pine, Sophora Root (Sophora flavescens Aiton), Japanese angelica root, safflower, Japanese barberry, betel nut (areca seed), tasmanian blue gum, Prunellae Spica, Akebia stem, Achyrantes root, Bupleurum root, Camellia sinensis, Licorice root (Glycyrrhiza), hop, Chrysanthemum, Senega root (Polygala senega), sesame, Cnidium rhizome (Cnidium officinal), Polygonum multiflorum, Pueraria root, Rosa maikwai H.Hara flower, saffron, rosemary, Rehmannia root, Tree mallow, Gambir, Chicory, Hierba Luisa (Lemon grass), yew, Artemisia capillaris, Borassus filabellifer, fennel, Common mallow, Rose fruit, Loasa urens, Polygala root, Kakocha, Polygonum multiflorum, Mexican ageratum, Valeriana fauriei, guarana, Centaurea calcitrapa, Cantharides, Catalpa ovata G. Don, pot marigold, Osmanthus fragrans var. aurantiacus, Japanese catnip (Schizonepeta tenuifolia Briq. var japonica Kitagawa), Pharbitis seed, Geranium thunbergii, cuachalalate, Greater celandine (Chelidonium majus), Kumaseba, Chinese nutgalls (Galla rhois), Great burdock achene (Arctii fructus), cilantro, Gleditsia japonica, Bupleurum root, Gardenia fruit, Japanese pepper, peony, Plantago seed, terculia foetida, Dichroa root, Cedron, Toad venom, Swertia japonica, Thuja orientalis L., Perilla herb, Sophora, Rhubarb, Piper angustifolium, Chanca piedra (Phyllanthus niruri), Clove, Picrasma ailanthoides, comfrey, betel nut (areca seed), Strychni Semen, Chaenomeles fruit, Leonurus japonicus, Lespedeza bicolor, Regro (Equisetum arvense), Colophony, cotton, Moutan bark, beinwell (Symphytum officinale L.), Mountain arnica, Sweet hydrangea (Hydrangea macrophylla var. thunbergii), Chinese caterpillar fungus (Cordyceps sinensis), Isodon japonicus Hara, Barley, orange, seaweed, cucumber, burdock, shiitake mushroom, Ransium domesticum, loquat, grape leaf, prune, sponge cucumber, Rosa maikwai H. Hara, lily and apple.

In addition, the following agents can be added to the composition of the present invention as long as they do not impair the effect of the present invention: lactic acid or its alkyl ester; organic acids such as succinic acid, malic acid and citric acid; protease inhibitor such as tranexamic acid; oils such as olive oil, squalane, liquid paraffin, isopropyl myristate, higher fatty acid and higher alcohol; polyhydric alcohol such as glycerin and propylene glycol; others e.g. surfactant such as ethylene oxide adducts of hydrogenated castor oil, humectant, thickener, antioxidant, ultraviolet absorber, algefacient, flavor, pigment, ethanol and water.

The composition for scalp and hair treatment of the present invention may be in any form as long as it is applicable externally to the scalp, such as liquid, emulsion, ointment, cream, gel and aerosol. The composition may be manufactured with appropriate base components conventionally used for manufacturing desired form. The composition of the present invention may be provided as medical, medicated cosmetic or cosmetic product.

The hair and scalp care composition of the present invention may be used for treating or preventing hair loss, baldness, alopecia, dandruff and/or itchy scalp. Non limiting examples of the condition to which the composition is applicable include treating or preventing male pattern baldness, diffuse alopecia which is mainly seen in women and alopecia areata.

The composition of the present invention may be topically applied or sprayed on the scalp. By the sake of the combination of the ingredients, the active ingredients are effectively adsorped transdermally.

The dosage of the hair and scalp care composition of the present invention may be determined according to age and sex of the subject and the extent of the hair loss or hair thinness to be treated. The amount may also vary depending on the dosage form of the composition. In general, for an adult male, the composition is applied in an amount that 0.0001-100 mg/day/kg body weight, preferably, 0.001-10 mg/day/kg body weight of the prostaglandin compound is applied. The amount may be given in divided doses 2-4 times per day.

### SYNTHESIS EXAMPLE 1

### 13,14-dihydro-15,15-trimethylenedioxy-20-ethyl-PGF_{2α} isopropyl ester (5)

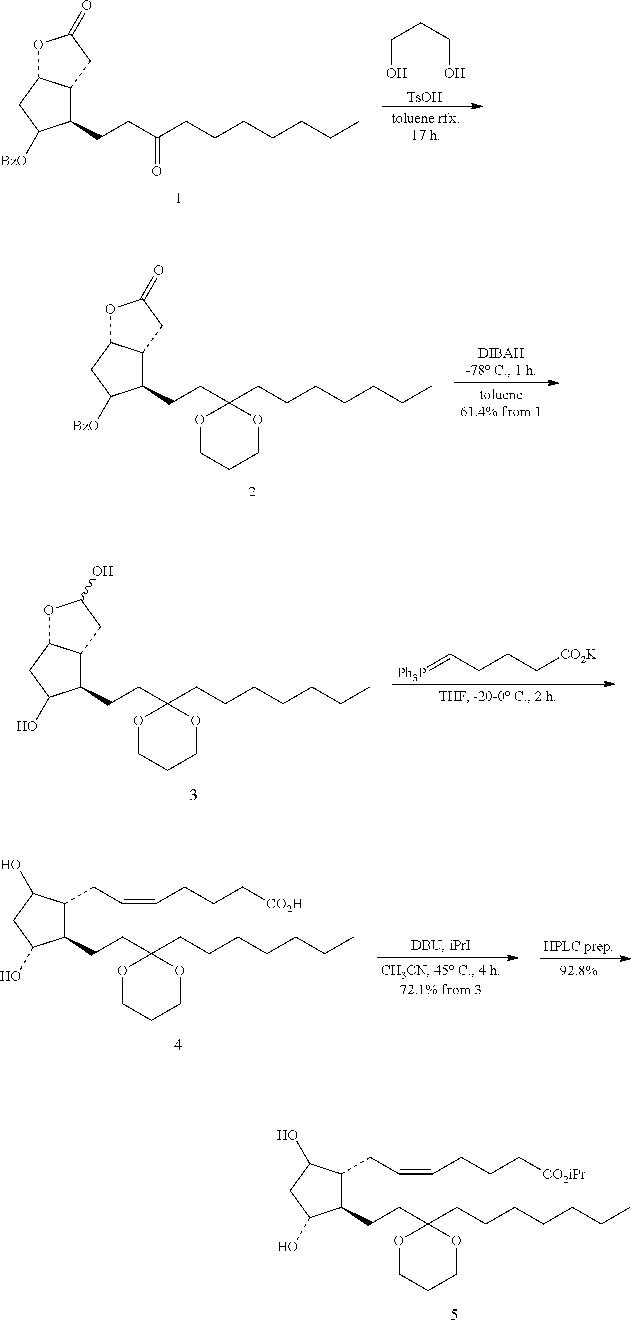

To the solution of compound 1 (510.0 mg, 1.273 mmol) in toluene (10.2 ml), 1,3-propanediol (0.92 ml, 12.73 mmol) and a catalytic amount of p-toluene sulfonic acid were added and the mixture was heated for 17 hours under reflux. After that, the reaction was left stood until it was cooled to room temperature, and washed with saturated aqueous sodium bicarbonate and saturated aqueous sodium chloride. The organic phase was dried with magnesium sulfate and evaporated under reduced pressure. The residue was purified by means of silica gel column chromatography (Merck 7734, Hexane:ethyl acetate=3:2) to give compound 2 (581.3 mg).

The solution of compound 2 (580.0 mg, 1.265 mmol) in toluene (11.6 ml) was cooled to −78° C., 1.5M-DIBAH (in toluene, 2.95 ml, 4.427 mmol) was added dropwise thereto and the mixture was stirred for 1 hour, and then, methanol (1.79 ml) was added dropwise to the resulting mixture. Saturated aqueous Rochelle salt (100 ml) was added thereto and the mixture was vigorously stirred for 30 minutes. The resulting mixture was extracted with ethyl acetate, and the organic layer was washed with saturated salt water, dried with magnesium sulfate and evaporated under reduced pressure. The residue was purified by means of silica gel column chromatography (Merck 7734, Hexane:ethyl acetate=1:9-0:10) to give compound 3 (275.2 mg, yield 61.4% from 1).

To the dispersion of (4-carboxybuthyl)triphenyl phosphonium bromide (1.346 g, 3.038 mmol) in THF (6 ml), 1M-potassium t-butoxide in THF (6.07 ml, 6.07 mmol) at 0° C. was added. The reaction was stirred for 1 hour at room temperature and then cooled to −20° C. Compound 3 (269.2 mg, 0.7594 mmol) in THF (7 ml) was added dropwise thereto and stirred for 2 hours at −20-0° C. Ice cold water was added to the reaction, THF was removed by evaporation evaporated under reduced pressure. To the concentrated residue at 0° C., ice cold 1N aqueous hydrochloric acid was added dropwise to adjust the solution to pH 4.

The solution was extracted with ethyl acetate and the organic layer was washed with saturated aqueous sodium chloride, dried with magnesium sulfate and evaporated under reduced pressure. The residue was added with ether and stirred for 17 hours at room temperature and then, filtrated with celite. The filtrate was evaporated under reduced pressure to give crude compound 4.

Compound 4 (0.7594 mmol) in acetonitrile (7.6 ml) was added with DBU (0.45 ml, 3.038 mmol), isopropyl iodide (0.30 ml, 3.038 mmol) and stirred for 4 hours at 45° C. The reaction mixture was evaporated under reduced pressure. The residue was added with water and extracted with ethyl acetate. The organic layer was washed with saturated aqueous sodium chloride solution, dried with magnesium sulfate and evaporated under reduced pressure. The residue was purified by means of silica gel column chromatography (Merck 9385, hexane:ethyl acetate=2:3) to give 727.2 mg of the desired product (yield 72.1% from 3). Thus obtained compound 4 (carboxylic acid, 259.0 mg) was further purified by separation HPLC to give compound 5 (isopropyl ester, 240.3 mg, HPLC purification yield 92.8%).

¹H-NMR spectrum (200 MHz,CDCl₃) of compound 5: δ5.57-5.14(2H, m), 5.01(1H, sept, J=6.2 Hz), 4.17(1H, bs), 3.97(1H, bs), 4.00-3.78(4H, m), 2.76(1H, d, J=6.2 Hz), 2.29(2H, t, J=7.5 Hz), 2.44-2.06 (5H, m,), 1.88(2H, bt,), 1.93-1.18(22H, m), 1.23(6H, d, J=6.2 Hz), 0.89(3H, t, J=6.8 Hz)

### SYNTHESIS EXAMPLE 2

### 13,14-dihydro-15,15-dimethoxy-20-ethyl-PGF_{2α} isopropyl ester (10)

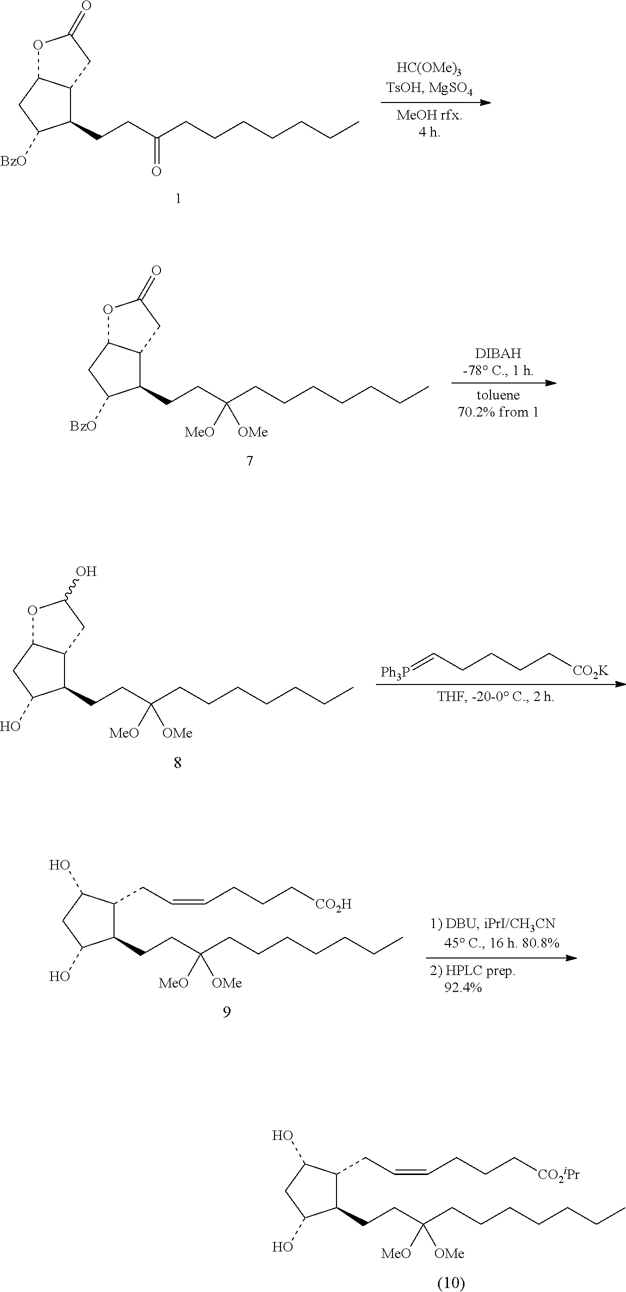

To the solution of compound 1 (797.8 mg, 2.002 mmol) in methanol (2.4 ml), a catalytic amount of p-toluene sulfate, methyl orthoformate(2.19 ml, 20.02 mmol) and unhydrous magnesium sulfate (1.20 g, 10.01 mmol) were added and heated under reflux for 4 hours. The reaction was cooled and added with sodium hydrogen carbonate, and filtered with celite. The filtrate was evaporated under reduced pressure and the residue was purified by means of silica gel column chromatography (Merck 7734 g, hexane:ethyl acetate=3:2) to give compound 7 (884.3 mg, yield 98.9%).

The solution of compound 7 (767.5 mg, 1.719 mmol) in toluene(15.4 ml) was cooled to −78° C., 1.5M-DIBAH (in toluene, 4.0 ml, 6.016 mmol) was added dropwise thereto and the mixture was stirred for 1 hour. Then, methanol was added dropwise to the reaction and the reaction was heated to room temperature. Saturated aqueous Rochelle salt (150 ml) was added thereto and the mixture was vigorously stirred for 30 minutes. The resulting mixture was extracted with ethyl acetate, and the organic layer was washed with saturated salt water, dried with magnesium sulfate and evaporated under reduced pressure. The residue was purified by means of silica gel column chromatography (Merck 9385, hexane:ethyl acetate=1:9) to give compound 8 (415.8 mg, yield 70.2%).

To the dispersion of (4-carboxybuthyl)triphenyl phosphonium bromide (1.250 g, 2.819 mmol) in THF, 1M-potassium t-butoxide in THF (5.64 ml, 5.64 mmol) at 0° C. was added. The reaction was stirred for 1 hour at room temperature and then cooled to −20° C. Compound 8 (242.8 mg, 0.7048 mmol) in THF (4 ml) was added dropwise thereto and stirred for 2 hours at −20-0° C. Ice cold water was added to the reaction, and THF was removed by evaporation under reduced pressure. To the residue at 0° C., ice cold 1N aqueous hydrochloric acid was added dropwise to adjust the solution to pH 5. The solution was extracted with ethyl acetate and the organic layer was washed with saturated aqueous sodium chloride, dried with magnesium sulfate and evaporated under reduced pressure. The residue was added with ether and stirred for 17 hours at room temperature and then, filtrated with celite. The filtrate was evaporated under reduced pressure to give crude compound 9 (carboxylic acid).

To the solution of compound 9 (0.7048 mmol) in acetonitrile (7 ml), DBU (0.42 ml, 2.819 mmol), isopropyl iodide (0.28 ml, 2.819 mmol) were added and the mixture was stirred for 16 hours at 45° C. The reaction mixture was evaporated under reduced pressure. The residue was added with water and extracted with ethyl acetate. The organic layer was washed with saturated aqueous sodium chloride, dried with magnesium sulfate and evaporated under reduced pressure. The residue was purified by means of silica gel column (Merck 9385, hexane:ethyl acetate=1:2) to give compound 10(268.0 mg, yield 80.8% from 8).

Compound 10 obtained as above (total 370 mg) was further purified by separation HPLC to give purified compound 10 (341.9 mg, HPLC purification yield 92.4%).

¹H-NMR spectrum (200 MHz,CDCl₃) of compound 10: δ5.54-5.13(2H, m), 5.00(1H, sept, J=6.2 Hz), 4.18(1H, bs), 3.95(1H, bs), 3.16(6H, s), 2.66(1H, d, J=6.4 Hz), 2.29(2H, t, J=7.3 Hz), 2.48-2.06(5H, m), 1.89(2H, bt), 1.79-1.17(20H, m), 1.23(6H, d, J=6.2 Hz), 0.89(3H, t, J=6.8 Hz)

### SYNTHESIS EXAMPLE 3

### 13,14-dihydro-15,15-ethylenedioxy-17-phenyl-18,19,20-trinor-PGF_{2α} isopropyl ester (12)

Compound 12 was prepared from compound 11 in a same manner as Synthesis example 1.

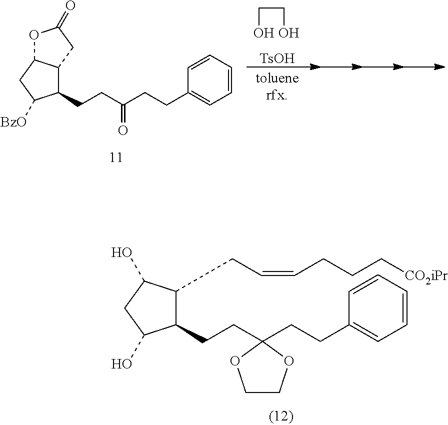

¹H-NMR spectrum (200 MHz, CDCl₃) of compound 11:δ8.04-7.93(2H, m), 7.63-7.38(3H, m), 7.35-7.11(5H, m), 5.21-5.03(2H, m), 2.98-2.24(11H, m), 2.12-1.98(1H, m), 1.80-1.50(2H, m)

¹H-NMR spectrum (200 MHz, CDCl₃) of compound 12:δ7.35-7.12(5H,m), 5.56-5.35(2H, m), 5.00(1H, sept, J=6.2 Hz), 4.15(1H, bs), 3.96(4H, s), 3.92(1H, bs), 3.18(1H, bd), 2.86(1H, bd), 2.75-2.63(2H, m), 2.28(2H, t, J=7.3 Hz), 2.46-1.15(17H, m), 1.22(6H, d, J=6.2 Hz)

### SYNTHESIS EXAMPLE 4

### 13,14-dihydro-15,15-ethylenedioxy-20-ethyl-PGF2α ethyl ester (15)

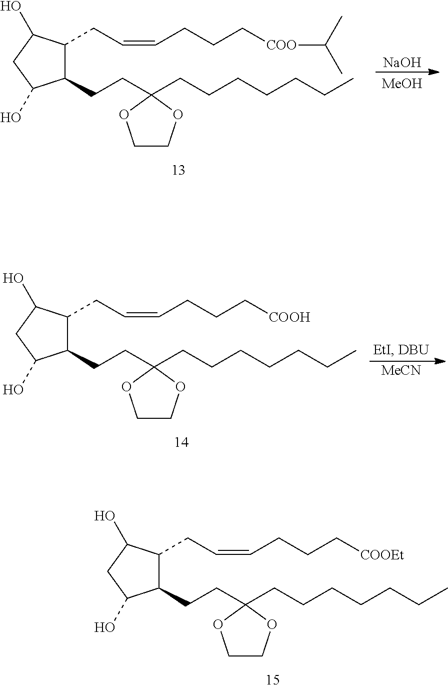

To the solution of compound 13 (9.18 g, 19.59 mmol) in methanol (91.8 ml), 8N-aqueous sodium hydroxide (24.49 ml) was added at 0° C. The reaction mixture was stirred for 3 hours at room temperature, and then acidified with 6N-hydrochloric acid at 0° C. The mixture was extracted with ethyl acetate (100 ml+50 ml). The organic layer was washed with saturated aqueous sodium chloride (100 ml×2), dried over anhydrous magnesium sulfate. The extract was evaporated under reduced pressure to obtain crude acid 14 as oil.

To the solution of crude acid 14 and 1,8-diazabicyclo[5.4.0]undec-7-ene (11.72 ml) in acetonitrile (60 ml), ethyl iodide (6.27 ml) was added dropwise at 0° C. The reaction mixture was stirred at 45° C. for 17 hours, then cooled to room temperature, and evaporated. To the residue, water (100 ml) was added. The mixture was extracted with ethyl acetate (100 ml×2). The organic layer was washed with 0.1N-hydrochloric acid, saturated aqueous sodium bicarbonate (100 ml) and saturated aqueous sodium chloride (100 ml). The extract was dried over anhydrous magnesium sulfate and evaporated. The residue was purified by two times of silica gel column chromatography (Merck 7734, 220 g, hexane:ethyl acetate=2:3, →BW-300, 210 g, hexane:2-Propanol=6:1) to obtain ethyl ester 15 (8.60 g, 18.92 mmol, 96.6% from 13) as a colorless oil.

¹H-NMR (200 MHz in CDCl₃, TMS=0 ppm) of the compound 15: δ 5.58-5.29(2H, m), 4.15(1H, brs), 4.13(2H, q, J=7.1 Hz), 3.97(1H, brs), 3.94(4H, s), 2.80-2.70(1H, br), 2.49-2.36(1H, m), 2.32(2H, t, J=7.4 Hz), 2.36-2.15(4H, m), 1.90-1.83(2H, m), 1.83-1.12(20H, m), 1.26(3H, t, J=7.1 Hz), 0.88(3H, t, J=6.5 Hz).

### EXAMPLES

The present invention will be explained in more detail by using examples. Those examples, however, should not be used for restricting the scope of the present invention.

The scalp and hair treatment lotions were prepared with ingredient shown in Table 1 and their effects on preventing dandruff and itchy scalp were evaluated. In the following example, the amounts of the ingredients are represented as per cent by weight (dry weight) based on the total weight of the composition unless otherwise indicated.

### 1) Preparation of Lotion

Ammonium glycyrrhizate, nicotinamide, 13,14-dihydro-15,15-ethylenedioxy-20-ethyl-PGF2α ethyl ester (hereinafter, referred as “PG compound A”), DL-α-tocopherol acetate, L-menthol and flavor were added to ethanol to give an alcohol component. Lactic acid, sodium lactate, dipropylene glycol, polyoxyethylene hydrogenated caster oil, and colorant were added to purified water to give an aqueous component. The alcohol and aqueous components were mixed, stirred and filtered to give test composition. The examples 2, 4, 6, 7 and 8 are comparative examples.

TABLE 1

| Example | 1 | 2 | 3 | 4 | 5 | 6 | 7 | 8 |
|---|---|---|---|---|---|---|---|---|
| PG compound A | 0.1 | 0.1 | 0.01 | 0.01 | 0.001 | 0.001 | — | — |
| L-menthol | 0.2 | — | 0.2 | — | 0.2 | — | 0.2 | — |
| Ammonium | 0.2 | 0.2 | 0.2 | 0.2 | 0.2 | 0.2 | 0.2 | 0.2 |
| Glycyrrhizate | | | | | | | | |
| POE hydrogenated | 0.5 | 0.5 | 0.5 | 0.5 | 0.5 | 0.5 | 0.5 | 0.5 |
| caster oil | | | | | | | | |
| nicotinamide | 0.1 | 0.1 | 0.1 | 0.1 | 0.1 | 0.1 | 0.1 | 0.1 |
| DL-α-tocopherol | 0.1 | 0.1 | 0.1 | 0.1 | 0.1 | 0.1 | 0.1 | 0.1 |
| acetate | | | | | | | | |
| colorant | Q.S. | Q.S. | Q.S. | Q.S. | Q.S. | Q.S. | Q.S. | Q.S. |
| flavor | Q.S. | Q.S. | Q.S. | Q.S. | Q.S. | Q.S. | Q.S. | Q.S. |
| lactic acid | Q.S. | Q.S. | Q.S. | Q.S. | Q.S. | Q.S. | Q.S. | Q.S. |
| sodium lactate | Q.S. | Q.S. | Q.S. | Q.S. | Q.S. | Q.S. | Q.S. | Q.S. |
| ethanol | 70.0 | 70.0 | 70.0 | 70.0 | 70.0 | 70.0 | 70.0 | 70.0 |
| purified water | balance | balance | balance | balance | balance | balance | balance | balance |
| dipropylene | 2.0 | 2.0 | 2.0 | 2.0 | 2.0 | 2.0 | 2.0 | 2.0 |
| glycol | | | | | | | | |
| itchy scalp | 0 | 0.6 | 0 | 0.6 | 0 | 0.8 | 0.2 | 0.8 |
| dandruff | 0 | 0.4 | 0 | 0.4 | 0 | 0.4 | 0.2 | 0.6 |
| sense of use | 4.4 | 3.0 | 4.2 | 3.0 | 4.2 | 2.8 | 3.8 | 2.0 |

### (2) Evaluation of Effects on Preventing Dandruff and Itchy Scalp, and Sense of Use.

Males complaining of dandruff and itchy scalp were received treatment with the compositions of examples 1-8 and the effects on preventing dandruff and itchy scalp were evaluated. The sense of use was also evaluated. This study was conducted with 5 people per group.

2-3 ml of the composition was applied to the scalp of each test subject twice a day for one month. During this period, the test subject washed his hair once a day with a shampoo containing no medicating ingredient. At the end of the treatment period, dandruff of the test subject was collected by a suction apparatus and the protein amount of the dandruff was measured. The amount of dandruff was evaluated according to the criteria shown below and the average score is shown in Table 1. In addition, the test subject evaluated itching on the scalp and sense of use according to the criteria shown below. The average score is shown in Table 1.

### Evaluation Criteria

### i) Itching of Scalp

3: Very itchy
2: Moderately itchy
1: Some itchy
0: Almost no itchy

### ii) Amount of the Dandruff

3: A lot of dandruff
2: Moderate dandruff
1: Some dandruff
0: Almost no dandruff
   iii) Sense of Use

Each person evaluated the sense of use of the composition and scored according to the following criteria and the average score is shown in Table 1.
5: very good
4: good
3: moderate
2: bad
1: very bad

### (3) Hair Growth Promoting Assay (Assay of Hair Revitalizing in Mice)

Experiment was carried out according to the method described by Ogawa et al. (Normal and Abnormal Epidermal Differentiation, edited by M. Seiji and I. A. Barstein, published by Todai Shuppan-kai, the reference is herein incorporated by reference). Male C3H mice (60 days old) were used. Hair on the back of each mouse was shaved into a size of about 2×4 cm. From the next day, test compositions were applied to the shaved area once a day every day. At day 18, the ratio of the area where hair regeneration was observed to total shaved area was evaluated and scored according to the criteria shown below. Three mice were used for each test composition and the average values are shown in Table 2.

### Evaluation Criteria

0: no hair regeneration was observed
1: hair regeneration was observed in less than 10% of the shaved area
2: hair regeneration was observed in about 30% of the shaved area
3: hair regeneration was observed in about 50% of the shaved area
4: hair regeneration was observed in more than 80% of the shaved area
   Results are shown in Table 2

TABLE 2

| Applied Composition | day 18 |
|---|---|
| control (75% ethanol) | 0 |
| Example 3 | 2.3 |
| Example 4 | 1.7 |
| Example 7 | 0 |
| Example 8 | 0 |

(4)Stability of 13,14-dihydro-15,15-ethylenedioxy-20-ethyl-PGF_{2α} Ethyl Ester (PG Compound A) in the Composition

The compositions of examples 1 and 2 were stored at room temperature for 2 months. After that, approximately 5g of each composition was weighed out and determined the weight precisely. Precisely 5 ml of octanophenone, inner standard, was added thereto to give test solution. Two micro litters of the test solution were injected to the column of liquid chromatograph and the stability of the PG compound A was determined. The ratio of peak area of PG compound to that of octanophene, inner standard (PG compound A/octanophene) was read from the chromatogram. The weight ratio (PG compound A/octanophene) was determined from the peak area ratio based on the preliminarily prepared calibration curve and the weight of the PG compound A in the composition was calculated. At the same time, the same compositions as above were stored for 2 months at 0° C. After that, the amount of PG compound A in the composition was determined in the same manner as above. The ratio of said compound stored at the room temperature to that stored at 0° C. was calculated with taking the amount of PG compound A stored at 0° C. as 100%.

### Measurement Conditions

LC System: LC-2010C (Shimadzu Corporation, Kyoto, Japan)
Detector: Ultraviolet Spectrometer
Wavelength: 254 nm
Column: CAPCELL PAK C18 MGII(S-3) (Shiseido Co., Ltd, Tokyo, Japan)
Operating Temperature: 40° C.
Mobile phase: Mixed solvent of dilute phosphate and acetonitrile
Flow speed: Adjusted so that the retention time of PG compound A was about 16 minutes.

Results are shown in Table 3.

TABLE 3

| Example | PG compound A (%) |
|---|---|
| 3 | 99.2 |
| 4 | 99.0 |

### FORMULATION EXAMPLES

Formulation examples are shown as follows:

### Formulation Example 1

### Object and Effects of the Formulation:

Preventing dandruff and itchy scalp. Providing exhilarant feelings.

TABLE 4

| ingredients | wt % |
|---|---|
| PG compound A | 0.05 |
| ethanol | 60 |
| dipropylene glycol | 2 |
| POE hydrogenated caster oil | 0.5 |
| lactic acid | Q.S. |
| sodium lactate solution | Q.S. |
| ammonium glycyrrhizate | 0.1 |
| nicotinamide | 0.1 |
| DL-α-tocopherol acetate | 0.1 |
| L-menthol | 0.2 |
| colorant | Q.S. |
| purified water | Balance |
| flavor | Q.S. |

### Formulation Example 2

### Object and Effects of the Formulation:

Preventing dandruff and itchy scalp. Providing exhilarant feelings.

TABLE 5

| Ingredients | wt % |
|---|---|
| PG compound A | 0.05 |
| ethanol | 75 |
| Polyoxyethylene polyoxy propylene | 1 |
| tetradecyl tetradecyl ether | |
| lactic acid | Q.S. |
| sodium lactate solution | Q.S. |
| panthenylethyl ether | 0.01 |
| benzyl nicotinate | 0.1 |
| nicotinamide | 0.2 |
| DL-α-tocopherol acetate | 0.05 |
| L-menthol | 1 |
| purified water | Balance |
| dimethyl ether | Q.S. |

### Formulation Example 3

### Object and Effects of the Formulation:

Preventing dandruff and itchy scalp. Providing exhilarant feelings.

TABLE 6

| Ingredients | wt % |
|---|---|
| PG compound A | 0.05 |
| methylpolysiloxane | 2 |
| ethanol | 7 |
| polyoxyethlene polyoxypropylene | 1 |
| decyltetradecyl ether | |
| talc | 5 |
| lactic acid | Q.S. |
| sodium lactate | Q.S. |
| benzyl nicotinate | 0.05 |
| nicotinamide | 0.05 |
| DL-α-tocopherol acetate | 0.2 |
| L-menthol | 0.3 |
| purified water | Balance |

### Formulation Example 4

### Object and Effects of the Formulation:

Preventing dandruff and itchy scalp. Preventing hair loss. Providing exhilarant feelings.

TABLE 7

| ingredients | wt % |
|---|---|
| PG compound A | 0.05 |
| ethanol | 80 |
| isostearilic alcohol | 2 |
| 1,3-buthlene glycol | 3 |
| POE hydrogenated castor oil | 1 |
| sodium lauryl sulfate | 0.3 |
| DL-malic acid | Q.S. |
| β-glycyrrhetinic acid | 0.2 |
| panthenylethyl ether | 0.1 |
| benzyl nicotinate | 0.1 |
| nicotinamide | 0.1 |
| DL-α-tocopherol acetate | 0.5 |
| aqueous decyltetradecyl dimethylmethyl | 5 |
| amine oxide (20%) | |
| L-menthol | 1 |

### Formulation Example 5

### Object and Effects of the Formulation:

Preventing dandruff and itchy scalp. Providing exhilarant feelings. Preventing hairs turning gray.

TABLE 8

| Ingredients | wt % |
|---|---|
| PG compound A | 0.05 |
| ethanol | 70 |
| isostearyl alcohol | 1 |
| trigryceryl-2-ethylhexanoate | 1 |
| lactic acid | Q.S. |
| sodium lactate solution | Q.S. |
| swertia japonica extract paste | 0.2 |
| β-glycyrrhetinic acid | 0.5 |
| panthenylethyl ether | 0.5 |
| benzyl nicotinate | 0.02 |
| nicotinamide | 0.1 |
| DL-α-tocopherol acetate | 0.1 |
| L-menthol | 0.5 |
| zanthoxylum fruit extract | 10 |
| purified water | Balance |
| flavor | Q.S. |

1.-11. (canceled)

12. A method for promoting hair growth of a subject in need thereof, which comprises applying an effective amount of a composition to the scalp and/or hair of the subject,
wherein the composition comprises a menthol derivative and a prostaglandin compound shown by the following formula (I): 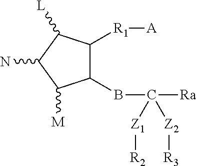
wherein L, M and N are hydrogen, hydroxy, halogen, lower alkyl, hydroxy(lower)alkyl, lower alkanoyloxy or oxo, wherein at least one of L and M is a group other than hydrogen, and the five-membered ring may have at least one double bond;
A is —CH₃, or —CH₂OH, —COCH₂OH, —COOH or a functional derivative thereof;
B is —CH₂—CH₂—, —CH═CH— or —C≡C—;
Z₁ and Z₂ are oxygen, nitrogen or sulfur;
R₂ and R₃ are optionally substituted lower alkyl, which is optionally linked together to form lower alkylene;
R₁ is a saturated or unsaturated bivalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkoxy, lower alkanoyloxy, cyclo(lower)alkyl, cyclo(lower)alkyloxy, aryl, aryloxy, heterocyclic group or heterocyclic-oxy group; lower alkoxy; lower alkanoyloxy; cyclo(lower)alkyl; cyclo(lower)alkyloxy; aryl; aryloxy; heterocyclic group; heterocyclic-oxy group.

13. The method as in claim 12, wherein the prostaglandin compound is represented by the formula (II): 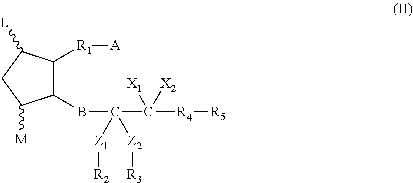
wherein L, M, A, B, Z₁, Z₂, R₁, R₂ and R₃ are the same as claim 2,
X₁ and X₂ are hydrogen, lower alkyl, or halogen;
R₄ is a single bond or lower alkylene; and
R₅ is lower alkyl, lower alkoxy, lower alkanoyloxy, cyclo(lower)alkyl, cyclo(lower)alkyloxy, aryl, aryloxy, heterocyclic group or heterocyclic-oxy group.

14. The method as described in claim 12, wherein said prostaglandin compound is 13,14-dihydro-15,15-ethylenedioxy-prostaglandin compound.

15. The method as described in claim 12, wherein said prostaglandin compound is 13,14-dihydro-15,15-ethylenedioxy-20-ethyl-prostaglandin compound.

16. The method as described in claim 12, wherein said prostaglandin compound is 13,14-dihydro-15,15-ethylenedioxy-20-ethyl-PGF_{2α} isopropyl ester.

17. The method as described in claim 12, wherein said prostaglandin compound is 13,14-dihydro-15,15-ethylenedioxy-17-phenyl-18,19,20-trinor-PGF_{2α} isopropyl ester.

18. The method as described in claim 12, wherein said prostaglandin compound is 13,14-dihydro-15,15-trimethylenedioxy-20-ethyl-PGF_{2α} isopropyl ester.

19. The method as described in claim 12, wherein said prostaglandin compound is 13,14-dihydro-15,15-dimethoxy-20-ethyl-PGF_{2α} isopropyl ester.

20. The method as described in claim 12, wherein said prostaglandin compound is 13,14-dihydro-15,15-ethylenedioxy-20-ethyl-PGF_{2α} ethyl ester.

21. The method as described in claim 12, wherein the composition comprises the prostaglandin compound in an amount of 0.0001-10 wt % (dry weight) and the menthol derivative in an amount of 0.001-5.0 wt % (dry weight) based on the total amount of the composition.

22. The method as described in claim 12, wherein R₁ of formula (I) is C6-10 hydrocarbon residue, and Ra of formula (I) is C1-8 hydrocarbon; and
the menthol derivative is L-menthol.

23. The method as described in claim 22, wherein the variables of formula (I) are as follows:
N is hydrogen;
L and M are hydrogen, —OH or oxo;
A is —COOH or its salt, ester or amide;
B is —CH₂—CH₂—; and
Z₁ and Z₂ are oxygen.

24. The method as described in claim 13, wherein R₁ of formula (II) is unsubstituted C6-10 hydrocarbon residue, and R₅ of formula (II) is a lower alkyl; and
the menthol derivative is L-menthol.

25. The method as described in claim 24, wherein the variables of formula (II) are as follows:
N is hydrogen;
L and M are hydrogen, —OH or oxo;
A is —COOH or its salt, ester or amide;
B is —CH₂—CH₂—; and
Z₁ and Z₂ are oxygen.

26. The method as described in claim 12, wherein the prostaglandin compound is a 13,14-dihydro-15,15-ethylenedioxy-20-ethyl-prostaglandin compound, and the menthol derivative is L-menthol.

27. The method as described in claim 26, wherein the 13,14-dihydro-15,15-ethylenedioxy-20-ethyl-prostaglandin compound is 13,14-dihydro-15,15-ethylenedioxy-20-ethyl-PGF2α ethyl ester or 13,14-dihydro-15,15-ethylenedioxy-20-ethyl-PGF2α isopropyl ester.
